(19) United States
(12) Patent Application Publication
(10) Pub. No.: US 2009/0220478 A1
(43) Pub. Date: Sep. 3, 2009

(54) Pharmaceutical active substance

(76) Inventor: Dirk Weickmann, Munchen (DE)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 11/629,342

(22) PCT Filed: Jun. 9, 2004
(86) PCT No.: PCT/DE04/01189

§ 371 Date: Apr. 6, 2009

(51) Int. Cl. A61K 38/46; A61K 38/02; A61P 35/00

(52) U.S. Cl. 424/94.6; 514/2

(57) ABSTRACT

Pharmaceutical active substance for treating tumors in the area of breast tissue (breast carcinoma) while using peptide toxin, a substance that acts in an antagonistic manner thereto, and/or a penetrating substance extracted from the venom of spiders of the family Lycosa tarantula.

For the ingestion of food in order to maintain its life, each animal depends on the offering given by the accessible plant and animal kingdom. However, not everything is suitable for consumption. In order to protect their own lives and to secure ingestion for themselves, several plants and animals use so called biogenic venoms adapted to their particular organisms and the specific needs thereof. In the course of long time development, these biogenic venoms have been established in the co-existence of different types of lives.

Due to this, every adult wild animal recognizes perilous plant and toxic animals of its natural environment.

Thereby, plants or animals can be primarily toxic by producing venoms or become secondary toxic when ingesting toxic substances from the living or lifeless environment.

In the history of mankind, utilization of these biogenic venoms began in primitive times leading to hunting down prey animals with venomed weapons. However, right from the beginning, the riskless application of these venoms required certain basic knowledge about its handling and efficacy. The further experiments conducted for deciphering the composition of the chemical configuration of biogenic venoms soon resulted in a well-directed search for specific active substances as the real initiators of the observed effects.

Particularly, the postulation raised by Paracelsus (1493-1541) concerning the isolation of active substances extracted from plants and contributing to the development of the iatrochemistry, i.e. the chemistry relating to the applications thereof, may have enhanced these efforts. Particularly, research made use of the skill of distilling substances resulting in a plurality of essential oils and volatile substances. However, for the isolation of other active substances or even the chemical deciphering thereof, the methods known at that time were insufficient.

First with the beginning of the 19^{th} century, the development of technical skills in chemistry proceeded far enough for ushering in the era of isolating pure active substances from biological materials.

For separating the demanded active substances from the associated materials, the differences in solubility of the examined substances in different solvents were used at first. Hereby, for example, there were observed differences in the distribution behaviour between two non-miscible fluid phases, in volatility and in chemical reactivity thereof.

The development of chromatography methods allowed an enormous improvement of separation techniques, the means of identifying active substances for fighting diseases, in the middle of the 20^{th} century. Based on the distribution between a mobile and a stationary fluid phase, on the adsorption, on the molecular sieve effects, on the ion exchange, on the affinity (particularly of proteins) for specific chemical compounds (e.g. enzyme substrates) and on the mobility of charged molecules in the electrical field, a plurality of novel separation techniques had been developed.

Actually, tumors as being the most perilous and formidable diseases of these days are being fought in a very radical and little environmentally friendly way. Simple characteristic keywords are steel, irradiation and chemotherapy.

On the one hand, this means that tumors, if reachable to some extent, are principally excised by using the steel of a knife, burned by wide-ranging irradiation or destroyed by a so-called chemotherapy with attacking and aggressive cytostatics also destroying healthy cells.

With both normal treatments using a scalpel and ionizing irradiation, a local restriction of the operating field is not possible. The destruction of healthy body cells is unavoidable. The undesired side effects of chemotherapy are well-known.

In contrast, it has been attempted to enable a cancer therapy on a subtle way deserving its name. For this purpose, one fell back on the rich treasure of the nature.

Hereto, among others, a lot of drugs being isolated from toxic creatures which are strongly effective substances in therapeutic doses are used.

From DE 199 61 141 A1, there is known a pharmaceutical active substance which has been found to be used for treating tumor diseases as comprising spider venoms of spiders belonging to the family Sicaridae as ingredients. A peptide toxin of the venom of this spider species, another substance that acts in an antagonistic manner thereto extracted from the venom and/or a combination of these substances are the main ingredients medically used.

This active substance can be used for treating tumor diseases as well as in tumor surgery in parallel or supportively and the residual tumor tissue can be destroyed. With this therapy, genetically modified body cells (tumor cells) can be destroyed as the relevant active substance recognizes the modified surface structure of such cells and kills them without causing complications. The total venom content of this spider species—quasi a cocktail of different substances—is not applicable for pharmaceutical purposes due to its lethal effect in even low doses.

This substance is even not appropriate for particular sorts of breast carcinoma (mamma carcinoma).

Therefore, it is an object of the active substance according to the present invention to effect—without causing complications—the killing of cancerous body cells from the area of the tissue of human mammary glands.

This object is achieved by an active substance characterized by one of the independent claims 1 to 4.

For solving the object underlying the active substance according to the independent claims, the peptide toxin and/or the substance that acts in an antagonistic manner thereto and/or the penetrating substance from the venom of Lycosa tarantula are used.

This spider species is also known as “wolf spider” having its name from the Italian city of Taranto (Tarentum) in Apulia. Therefore, it is often referred to as Lycosa tarentula. Both notations are used.

In 1370 a mysterious epidemic appeared close to this city, wherein, like common belief in these days, the only healing and recovery from supposed death was to dance in a wild manner until tumbling down (tarantella). For three hundred years, in each summer, this epidemic appeared which was attributed to the bite of a large hairy spider, viz. the wolf spider. First in 1600, the harmlessness of this spider species was found out.

Interestingly, it was disclosed later on that the reason for disease and sometimes also for death was the bite of another spider from the family Latrodectus (the Black Widow).

Lycosa tarantula is among the carnally biggest spiders in Europe and belongs to the family Lycosidae having several members almost all around the world.

This spider species does not trap its prey in webs but reaches them by running and the speed of its legs. It lives in a self-digged hole in soil which is covered with a layer of spider webs in winter times.

Lycosa tarantula is about 5 cm in size and cannot become perilous for mankind.

The peptide toxin and the substance that acts in an antagonistic manner thereto and/or the penetrating substance can be obtained by per se known fractioning methods for separation of proteins from the spider venom raw mixture (spider venom cocktail). Preferably, the peptide toxin and the substance that acts in an antagonistic manner thereto are obtained by gel chromatography, HPLC, affinity chromatography and/or ion exchange chromatography.

The substance that acts in an antagonistic manner thereto and/or the penetrating substance are preferably a phospholipase or a hyaluronidase or a combination of both substances.

Moreover, it is preferred that the peptide toxin and the substance that acts in an antagonistic manner thereto and/or the penetrating substance are present in such an amount as an pharmaceutically active substance that a destroying effect for tumor cells will be attained by the active substance. Furthermore, the required proportions are selected such that the peptide toxin shows none or only little toxic effects in the patient to be treated. Naturally, the amounts of the pharmaceutical active substances are hereby also to be adjusted to the type of the tumor to be treated and the physical, optionally also the psychic, conditions of the respective patient. The preliminary tests necessary for such an adjustment have to be carried out by a person skilled in the art due to its expertise and technical skills.

The amount of the penetrating substance is preferably selected to recognize the abnormal cells to a large extent and to selectively destroy tumor cells in combination with the peptide toxin wherein healthy cells are preserved as far as possible.

Hereby, a spatially and temporally controlled distribution of the active substance has to be ensured.

It is further preferred that the pharmaceutical active substance contains conventional carriers and excipients like antibiotics, antimycotics, antituberculotic agents, means against parasites, cytostatics, amino acids, wound healing enhancing enzymes and/or mitosis inhibiting active substances. Penicillin/streptomycin, polynyxin/gentamycin (5%), mitopodozide, vinca rosea lacaloids, bromelaina or bromelains are preferred.

The pharmaceutical active substance according to the present invention is used in a combination of the partial active substances described. However, it is also possible to use the partial active substances in specific cases in such a way that the specific effects of the substances are suited for therapeutic purposes. It is also possible to produce the partial active substances described in a recombinant form by using chemically synthetic methods or methods of genetic engineering. As customary for chemical substances, the present invention also comprises derivatives and salts of the substances provided by the present invention. For example, the peptide toxin can comprise one or more substitutions and/or deletions of amino acids wherein it has to be ensured that the medical effect according to the present invention will be maintained.

The extraction of the partial active substances described is carried out by methods common in the field of chemical engineering.

It has surprisingly be determined in longsome and detailed experiments that particular toxins in the active substance according to the present invention show special lysing effects in association with their respective molecular weights.

Those are the molecular weights of 18, 54, 108 and 124 (the unit is always measured in kDa referred to as kilo Dalton).

In certain cases, it is preferable to use the pure peptide venom extract of Lycosa tarantula rather as a solubilizer for the production of a pharmaceutical active substance from the toxin of spiders of the genus Loxosceles. Moreover, a part of the phospholidases from the total venom extracts of Lycosa tarantula can be advantageously combined with the Loxosceles toxins.

1. A pharmaceutical active substance for treating tumors in the area of breast tissue (breast carcinoma) containing in a pharmaceutically effective amount:
a) at least one peptide toxin as well as
b) at least one substance that acts in an antagonistic manner thereto and/or at least one penetrating substance,
wherein at least the peptide toxin is extracted from the venom of spiders of the family Lycosa tarantula and optionally the substance that acts in an antagonistic manner thereto and/or the penetrating substance is extracted from the venom of spiders of the family Lycosa tarantula and
c) wherein the molecular weight of the peptide toxin is 18 kDa.

2. A pharmaceutical active substance for treating tumors in the area of breast tissue (breast carcinoma) containing in a pharmaceutically effective amount:
a) at least one peptide toxin as well as
b) at least one substance that acts in an antagonistic manner thereto and/or at least one penetrating substance,
wherein at least the peptide toxin is extracted from the venom of spiders of the family Lycosa tarantula and optionally the substance that acts in an antagonistic manner thereto and/or the penetrating substance is extracted from the venom of spiders of the family Lycosa tarantula and
c) wherein the molecular weight of the peptide toxin is 54 kDa.

3. A pharmaceutical active substance for treating tumors in the area of breast tissue (breast carcinoma) containing in a pharmaceutically effective amount:
a) at least one peptide toxin as well as
b) at least one substance that acts in an antagonistic manner thereto and/or at least one penetrating substance,
wherein at least the peptide toxin is extracted from the venom of spiders of the family Lycosa tarantula and optionally the substance that acts in an antagonistic manner thereto and/or the penetrating substance is extracted from the venom of spiders of the family Lycosa tarantula and
c) wherein the molecular weight of the peptide toxin is 108 kDa.

4. A pharmaceutical active substance for treating tumors in the area of breast tissue (breast carcinoma) containing in a pharmaceutically effective amount:
a) at least one peptide toxin as well as
b) at least one substance that acts in an antagonistic manner thereto and/or at least one penetrating substance,
wherein at least the peptide toxin is extracted from the venom of spiders of the family Lycosa tarantula and optionally the substance that acts in an antagonistic manner thereto and/or the penetrating substance is extracted from the venom of spiders of the family Lycosa tarantula and
c) wherein the molecular weight of the peptide toxin is 124 kDa.

5. The pharmaceutical active substance according to any of claims 1 to 4, characterized in that
the pure peptide toxin extract from the venom of spiders of the family Lycosa tarantula is merely used as a solubilizer for the production of a pharmaceutical active substance from the venom of the spiders of the genus Loxosceles.

6. The pharmaceutical active substance according to any of the claims 1 to 4, characterized in that
a part of the phospholipases is combined with toxins from spiders of the genus Loxosceles.
